(19) United States
(12) Patent Application Publication
(10) Pub. No.: US 2026/0315229 A1
(43) Pub. Date: Oct. 15, 2026

(54) TRABECULAR STRUCTURE FOR FEMORAL AND TIBIAL AUGMENTS

(71) Applicant: BIORAD MEDISYS PRIVATE LIMITED, Pune (IN)

(72) Inventors: Jayjeet SHIVANAND BHOITE, Pune (IN); Ankita JALANDAR PATIL, Pune (IN); Debasish PRADHAN, Pune (IN); Pooja RAMCHANDRA MALI, Pune (IN)

(21) Appl. No.: 19/485,281

(22) PCT Filed: May 17, 2024
(86) PCT No.: PCT/IN2024/050581

§ 371 Date: Nov. 18, 2025

(30) Foreign Application Priority Data
May 18, 2023 (IN) 202321034826

(51) Int. Cl. A61F 2/30; A61F 2/38

(52) U.S. Cl. CPC A61F 2/30734; A61F 2/38; A61F 2002/30011; A61F 2002/30131; A61F 2002/30161; A61F 2002/30433; A61F 2002/30616; A61F 2002/30736; A61F 2002/3092; A61F 2002/3093

(57) ABSTRACT

The present invention discloses a trabecular structure for femoral and tibial augment. The trabecular structure comprises an augment (104). The augment (104) is disposed on the bone implant (102). The augment comprises a wall (204) and a 5 porous structure (202), at least one threaded hole (108) for a fastener (106), a pair of flat surfaces and at least one opening (110) for disposing the bone implant (102). The porous structure (202) enclosed inside of the wall (204). The porous structure (202) is made up of a plurality of cavities (214). The plurality of cavities (214) are form by cross connecting a plurality of straight ribs (216). The augment (104) 10 enables a growth of the bone through the porous structure (202).

### TECHNICAL FIELD

This invention is generally in the field of medical devices and specifically relates to orthopedic prosthesis. More particularly, the invention relates to an augment system that stabilize and align with femoral and tibial implant/support structures for use with a knee prosthesis.

### BACKGROUND

The natural human knee joint involves the distal end of the femur, the proximal end of the tibia and a meniscus bearing there in between. Knee replacement surgery methods and knee joint prostheses are known in the art. A typical knee joint prosthesis includes a femoral component implanted on the distal end of the femur, which articulates with a tibial component implanted on the proximal end of a tibia to replicate the function of a healthy natural knee. When such knee replacement surgery is performed, an incision is made to expose the knee joint in order to enable removal of both the proximal portion of the tibia and the distal portion of the femur, which creates surfaces upon which the tibial and femoral components of the knee prosthesis can be attached.

To ensure that the natural joint line is preserved in the joint replacement procedure, the distal portion of the femur and the proximal portion of the tibia may each be respected by an amount corresponding to the thicknesses of the femoral and tibial components, respectively, such that the effective overall lengths of the femur and tibia remain unchanged after implantation of the prosthetic components. However, in some cases the proximal tibia or distal femur may have severe degeneration, trauma, or other pathology which necessitates resection of more bone. The prior art prosthetic members have not been wholly satisfactory for knee implantation because they do not provide for the complexity involved when flexing the tibia relative to the femur. In such cases, augments may be used to effectively increase the thickness of the implanted component, thereby compensating for the additional thickness of the bone resection. The augment system can be used with an implant component i.e. femoral component and/or a tibial component and can offer versatility, flexibility, and structural support, while compensating for bone damage or other deficiencies of the tibia or femur. Depending on the amount of bone loss or deterioration, augments may be used with the implant components to increase the overall volume of implant component and compensate for the loss of bone in particular areas. Additionally, such augments may improve the fixation of implant components to the patient's tibia by reducing the amount of bone cement or other adhesive required to secure the tibial tray to the patient's tibia.

### SUMMARY

This summary is provided to introduce concepts related to an augment system that stabilize and align with femoral and tibial implant/support structures for use with a knee prosthesis. This summary is neither intended to identify essential features of the present invention nor intended to determine or limit the scope of the present invention.

Embodiment of the present disclosure introduces technological improvements as solutions to one or more of the above-mentioned technical problems recognized by the inventor(s).

In an embodiment of the present invention, the trabecular structure for femoral and tibial implant is disclosed. The trabecular structure comprises an augment. The augment is disposed on the bone implant. The augment comprises a wall and a porous structure. The porous structure enclosed inside of the wall. The porous structure is made up of a plurality of cavities. The plurality of cavities are form by cross connecting a plurality of straight ribs. The augment enables a growth of the bone through the porous structure.

Porus structure has 0.2 mm to thickness of augment with an average porosity of about 60 to 70%, and a pore size having range between 450 μm to 750 μm. The material of augment is Ti6Al4V, which has better strength and excellent corrosion resistance. Alternatively, material of augment may include Co—Cr—Mo alloy, SS316L, Ti—Ta alloy and is not limited to these materials only. Further, in case, material of Co—Cr—Mo alloy or SS316L is used, then, to prevent allergy due to presence of Nickel, coating comprising of Ti—Nb—N or Ti—N can be made us of. The U-shape on the posterior side of the tibial augment relieves the hamstring muscle/tendon.

These and other features and advantages of the present invention will become apparent from the following description of the invention that is provided in connection with the accompanying drawings and illustrated embodiments of the invention.

It will be appreciated that features of the present disclosure are susceptible to being combined in various combinations without departing from the scope of the present disclosure as defined by the below mentioned detailed description and drawings.

### BRIEF DESCRIPTION OF THE DRAWINGS

The foregoing detailed description of embodiments is better understood when read in conjunction with the appended drawings. For the purpose of illustrating the disclosure, there is shown in the present document example constructions of the disclosure; however, the disclosure is not limited to the specific apparatus or method disclosed in the document and the drawings.

The present disclosure is described in detail with reference to the accompanying figures. In the figures, the left-most digit(s) of a reference number identifies the figure in which the reference number first appears. The same numbers are used throughout the drawings to refer various features of the present subject matter.

Embodiment of the present disclosure will now be described, by way of example only, with reference to the following diagrams wherein:

FIG. 1A illustrates an assembly view of alignment between tibial implant and tibial augment with an exemplary embodiment of the present disclosure.

FIG. 1B illustrates exploded view of tibial implant components and tibial augment components with an exemplary embodiment of the present disclosure.

FIG. 2A illustrates anatomical shape of tibial augment in accordance with an exemplary embodiment of the present disclosure.

FIG. 2B illustrates trabecular structure of tibial augment in accordance with an exemplary embodiment of the present disclosure.

FIG. 3 illustrates distal femoral augment in accordance with an exemplary embodiment of the present disclosure.

FIG. 4 illustrates posterior femoral augment in accordance with an exemplary embodiment of the present disclosure.

FIG. 5A, FIG. 5B and FIG. 5C illustrates distal femoral augment with trabecular structure in different thickness in accordance with an exemplary embodiment of the present disclosure.

FIG. 6A and FIG. 6B illustrates posterior femoral augment with trabecular structure in different thickness in accordance with an exemplary embodiment of the present disclosure.

FIG. 7A, FIG. 7B and FIG. 7C illustrates full tibial augment with trabecular structure in different thickness with an exemplary embodiment of the present disclosure.

In the above accompanying drawings, a non-underlined number relates to an item identified by a line linking the non-underlined number to the item. When a number is non-underlined and accompanied by an associated arrow, the non-underlined number is used to identify a general item at which the arrow is pointing.

Further, the figures depict various embodiments of the present subject matter for purposes of illustration only. One skilled in the art will readily recognize from the following discussion that alternative embodiments of the structures and methods illustrated herein may be employed without departing from the principles of the present subject matter described herein.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although an apparatus for wall mounting shoe for self-climbing platform, similar or equivalent to those described herein can be used in the practice or testing of embodiments of the present disclosure, the exemplary, an apparatus for femoral and tibial implant is now described.

As used herein, the terms “proximal” and “distal” should be given their generally understood anatomical interpretation. The term “proximal” refers to a direction generally towards the torso of a patient and “distal” refers to the opposite direction of proximal, that is, against the torso of a patient. It should be understood that the use of the terms “proximal” and “distal” should be interpreted as if the patient were standing with the knee joint in extension even though the appliances described herein are generally used with the joint knee in flexion. The intention is to differentiate the terms “proximal” and “distal” from the terms “anterior” and “posterior”. As used herein, the terms “anterior” and “posterior” should be given their generally understood anatomical interpretation. Thus, “posterior” refers to a rear portion of the patient, e.g., a rear portion of the knee. Similarly, “anterior” refers to a front part of the patient, e.g. a front part of the knee. Thus, “posterior” refers to the opposite direction of “anterior.” Similarly, the term “lateral” refers to the opposite direction of “medial.”

The person skilled in the art will recognize many variations, alternatives, and modifications of the embodiment of the present disclosure. The person skilled in the art will recognize that the “augment” can be a “tibial augment” or “distal femoral augment” or “posterior femoral augment”. The “augment” word in the draft can be used for “tibial augment” or “distal femoral augment” or “posterior femoral augment”. Also, the person skilled in the art will recognize that the “bone implant” can be a “tibial implant” or “femoral implant”. The “bone implant” word in the draft can be used for “tibial implant” or “femoral implant”. It should be understood that this invention is not limited to the particular methodology, protocols, and the like, described herein and as such may vary. The terminology used herein is for the purpose of describing particular embodiment only and is not intended to limit the scope of the present invention, which is defined solely by the claims.

Various modifications to the embodiment will be readily apparent to those skilled in the art and the generic principles herein may be applied to other embodiments. For example, although the present disclosure will be described in the context of trabecular structure for femoral implant and tibial implant, one of ordinary skill in the art will readily recognize that an apparatus can be used for other bone implants also. Thus, the present disclosure is not intended to be limited to the embodiments illustrated but is to be accorded the widest scope consistent with the principles and features described herein.

The present disclosure provides technical solutions to the technical problems identified in the prior art. In the prior art, an augment system can include two or more stackable augments configured to attach with an implant component and be located between the implant and a resected surface of a tibia/femur. The augment system can include plates of variable thickness and plates formed of different materials. The augment system can facilitate restoration of the anatomic joint line and address bone deficits on all or some of a proximal surface of a patient's tibia. Furthermore, in some cases, a surgeon may use a second kind of augment, such as an augment having a generally cone-shaped outer profile corresponding to the generally cone-shaped bone defect typically encountered within the tibia.

Accordingly, there is a need for augments which do not increase the complexity of the reconstruction and overcome the prior art problems. This invention is therefore directed towards providing an improved augment assembly having a porous trabecular structure for femur and tibial augments that enable enhanced/superior bone integration and stability. Further, improved augment assemble is relatively simple in design and structure, and is highly effective for its intended purpose. The augment is configured to bear structural stability and to improve osteointegration.

In an embodiment of the present invention, the porous trabecular structure for femoral and tibial implant is disclosed. The trabecular structure comprises an augment which is disposed on the bone implant. The augment comprises a wall and a porous structure, at least one threaded hole for a fastener, groove and at least one opening for disposing the bone implant.

The porous trabecular structure which is made up of a plurality of cavities is enclosed inside of the wall. The plurality of cavities are formed by cross connecting a plurality of straight ribs. The augment enables a growth of the bone through the porous structure.

The augment comprises at least one threaded hole for a fastener. The fastener is configured to constrain the motion of the augment with respect to the bone implant. The augment comprises a first surface and a second surface. The first surface comes in contact with the bone implant and the second surface comes in contact with a cut surface of the bone. The augment comprises at least one opening for disposing the bone implant. The augment comprises groove on posterior side. The groove is configured to provide space for the hamstring muscle.

The tibial augment comprises an anatomical shape. The anatomical shape comprises a curved surface between first surface and the second surface. The curved surface is convexly protruding to the outside of the augment to match with the anatomical tibial bone shape. The curved surface comprises a predetermined angle of inclination. The predetermined angle of inclination depends on knee anatomy of a patient.

The material of the augment is TI6Al4V. The predefined thickness of the augment is 0.2 mm. The density of the augment varies from 60 to 70%, The pore size of the augment is 450 μm to 650 μm. The height of the augment from the first surface to the second surface is varies from 2 mm to 25 mm. A plurality of augments are assembled on the bone implant to provide bone integration and stability.

The present invention comprises a tibial augment having an anatomical shape of a structure that allows lost bone to be reinforced as much as possible through the augment having the shape conforming to the anatomical shape of tibial bone.

The present invention comprises a tibial augment having an anatomical shape capable of increasing the life of an implant by preventing damage of the implant or bone through a normal load distribution.

The present invention comprises a knee joint augment that will provide enhanced/superior bone integration and stability. A plurality of augments are assembled on the bone implant to provide more bone integration and stability.

The present invention comprises a femoral augment with interlocking features with trabecular structure on the surface. The invention further discloses tibial augments with bone anatomical shape with trabecular structure on the surface adapted to provide better/superior bone integration and stability as compared with cemented/uncemented augments.

It should be noted that the above advantages and other advantages will be better evident in the subsequent description. Further, in the subsequent section, the present subject is better explained with reference to the figures. In order to maintain consistency and brevity of reading, the all the figures from 1A, 1B, 2A, 2B, 3, 4, 5A, 5B, 5C, 6A, 6B, 7A, 7B, 7C are explained jointly. Further, the following table lists of nomenclature and numberings are used in the figure to illustrate the invention and the nomenclature is further used to describe in the invention the subsequent paragraph.

| Nomenclature | Number | Nomenclature | Number |
|---|---|---|---|
| Bone Implant | 102 | Threaded hole for Fastner | 108 |
| Tibial augment | 104 | Opening for tibial implant | 110 |
| Wall | 204 | First surface | 208 |
| Porous Structure | 202 | Second surface | 210 |
| Plurality of cavities | 214 | Groove | 112 |
| Plurality of straight ribs | 216 | Curved surface | 212 |
| Fastner | 106 | Distal femoral augment | 304 |
| Tibial augment and | 100 | Posterior femoral augment | 306 |
| implant assembly | | | |
| Distal femoral | 304-1 | Posterior femoral augment | 306-1 |
| augment 5 mm | | 5 mm | |
| Distal femoral | 304-2 | Posterior femoral augment | 306-2 |
| augment 10 mm | | 10 mm | |
| Distal femoral | 304-3 | Tibial augment 10 mm | 104-2 |
| augment 15 mm | | | |
| Tibial augment 5 mm | 104-1 | Tibial augment 15 mm | 104-3 |

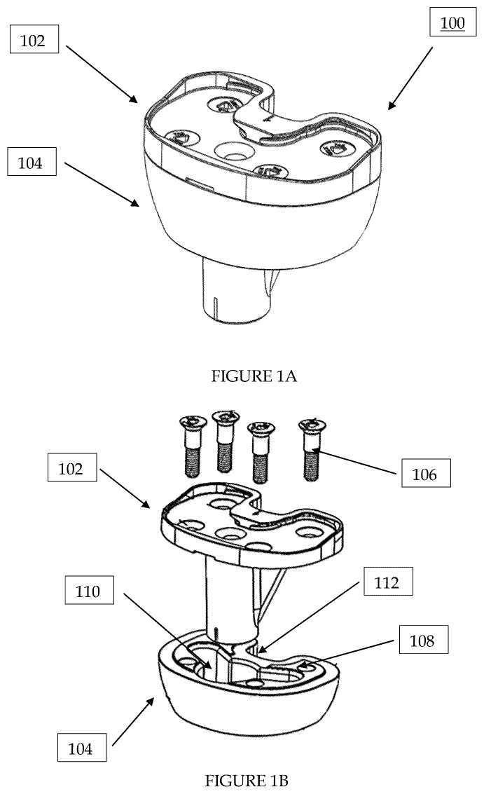

FIG. 1A illustrates an assembly view of alignment between tibial implant 102 and tibial augment 104 with an exemplary embodiment of the present disclosure. The assembled view 100 represents alignment of tibial implant 102 and tibial augment 104.

FIG. 1B illustrates exploded view of the tibial augment 104 and tibial implant 102 with an exemplary embodiment of the present disclosure. The tibial augment 104 comprises a threaded hole 108 for a fastener 106. The fastener 106 is configured to constrain the motion of the augment 104 with respect to the bone implant 102. The tibial augment 104 comprises an opening 110 for disposing the bone implant 102. The tibial augment comprises groove 112 on posterior side. The groove 112 is configured to provide space for the hamstring muscle.

A threaded hole 108 for a fastener 106 from tibial implant 102 are recessed downward by a predetermined depth to assemble tibial tray with the tibial augment 104. This predetermined depth depends on thickness of trabecular structure provided onto tibial augment 104. In other words, distal end of fastener 106 is threaded and are inserted within the opening 104 and thereby gets fit inside a threaded hole 108 of tibial augment 104. This enables alignment between tibial implant 102 and tibial augment 104. Additionally, this provides an added advantage wherein neither tibial implant 102 nor tibial augment 104 gets dislocated and allows damaged knee to function similarly like that of human natural knee. In addition to this, an opening 110 is provided onto tibial augment 104 in which keel of tibial implant 102 gets placed to avoid dislocation of tibial implant 102 and tibial augment 104.

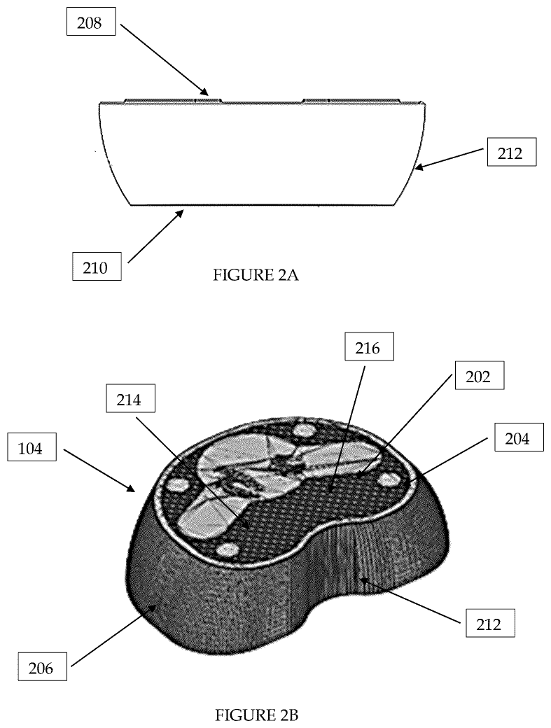

FIG. 2A illustrates the anatomical shape of the tibial augment 104 in accordance with an embodiment of present disclosure. The tibial augment comprises first surface 208 and a second surface 210. The first surface 208 comes in contact with the tibial bone implant 102 and the second surface 210 comes in contact with a cut surface of the tibial bone. The outer surface of tibial augment 104 may form a curved surface 212 convexly protruding to the outside of the tibial augment 104 that goes from the bottom surface 210 to the top surface 208 and matches with the anatomical bone shape of that of tibia. In one exemplary embodiment tibial augment 104 is utilized while in another exemplary embodiment tibial half augment may also be used. The choice of tibial augment 104 i.e. half or full depends on percentage of damage and percentage of resection that patient knee has undergone to enable functioning of knee prosthesis similar like that of natural human knee.

Referring to FIG. 2B illustrates a trabecular structure in tibial augment 104. The tibial augment 104 comprises a wall 204 and a porous structure 202. The porous structure 202 enclosed inside of the wall 204. The porous structure 202 is made up of a plurality of cavities 214. The plurality of cavities 214 are form by cross connecting a plurality of straight ribs 216. The augment 104 enables a growth of the bone through the porous structure 202.

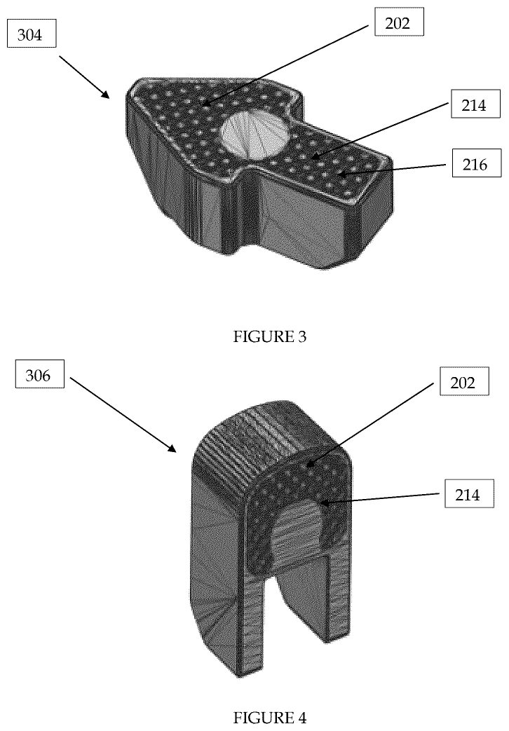

Referring to FIG. 3 and FIG. 4 illustrates a interlocking femoral augment is disclosed. The present interlocking femoral augment comprise a distal femoral augment 304 and a posterior femoral augment 306 which avoids rotation in femur. The interlocking distal femoral augment 304 and the posterior femoral augment 306 are arranged and aligned in conjunction with femoral implant. Trabecular structure with 0.2 mm to full thickness of each of the distal femoral augment 304 and the posterior femoral augment 306 compensates the bone defect volume. Furthermore, 0.2 mm to full thickness of augment protruding on augment top surface facilitates interlocking between femoral component and distal femoral augment 304 and a posterior femoral augment 306 to avoid rotation and provide grip. Additionally, shape & geometry of the femoral augment facilitates compact fitment with the femoral implant to facilitate functioning of knee prosthesis similarly like that of natural human knee without causing any difficulty.

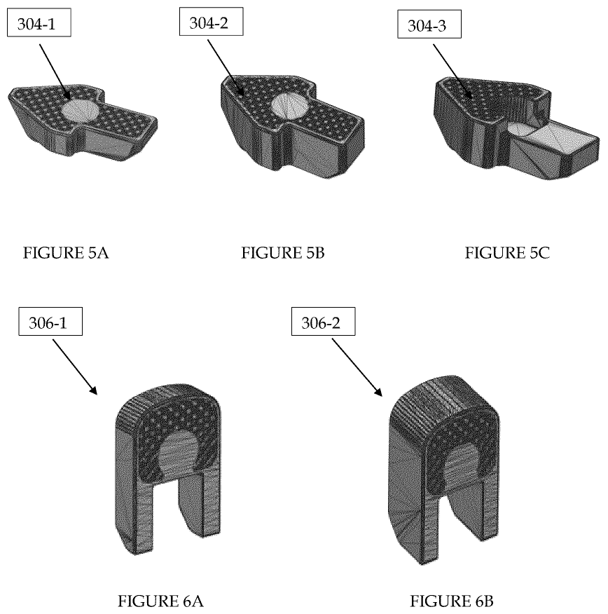

Referring to FIG. 5A, FIG. 5B and FIG. 5C, illustrates distal femoral augment 304 with trabecular structure of height 5 mm, 10 mm and 15 mm respectively.

FIGS. 6A and 6B discloses posterior femoral augment 306 with trabecular structure of sizes 5 mm and 10 mm respectively. In another exemplary embodiment, trabecular structure of other/varied size in reference to distal femoral augment 304 and posterior femoral augment 306 may be utilized and is dependent on patient's knee anatomy.

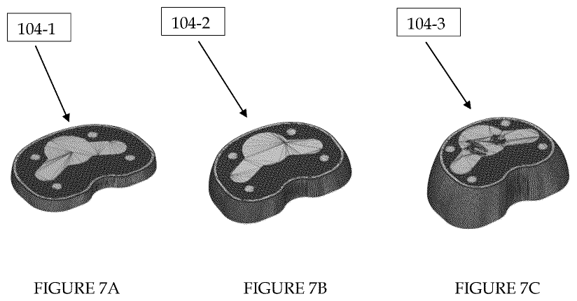

FIG. 7A, FIG. 7B and FIG. 7C, illustrates tibial augment 104 with trabecular structure of sizes 5 mm, 10 mm and 15 mm thickness respectively. The height of the augment (104) from the first surface 208 to the second surface 210 varies from 2 mm to 25 mm. The tibial augment 104 are aligned with tibial implant 102. Further, trabecular structure of varied size may also be utilized in accordance with patient's knee anatomy. In present invention, tibial augments be it full or partial are designed and configured to accommodate implant features. This is ensured by providing a threaded hole 108 which in illustrated FIG. 1B are four in number and may vary in accordance with patient's knee anatomy. Further, to accommodate implant features in tibial augment 104, an opening 110 is provided for holding tibial implant 102.

Tibial augment is available with bone anatomical shape with trabecular structure on the surface adapted to provide better/improved/superior bone integration and stability as compared with cemented/uncemented augments. Trabecular structure has 0.2 mm to full thickness of augment with an average porosity of about 60 to 70%, and a pore size having range between 450 μm to 750 μm. The material of augment is Ti6Al4V, which has better strength and excellent corrosion resistance. Alternatively, material of augment may include Co—Cr—Mo alloy, SS316L, Ti—Ta alloy and is not limited to these materials only. Further, in case, material of Co—Cr—Mo alloy or SS316L is used, then, to prevent allergy due to presence of Nickel, coating comprising of Ti—Nb—N or Ti—N can be made us of. The U-shape on the posterior side of the tibial augment relieves the hamstring muscle/tendon. Further, shape of tibial augment may vary and is in accordance with patient's knee anatomy.

It is observed that augments are non-stackable. The chances of dislocation has been eliminated as the trabecular structure on augment will provide more bone integration and stability. The present invention has both technical as well as economic significance.

While a particular embodiment of the invention has been illustrated and described, modifications thereof will readily occur to those skilled in the art. It is understood that the various embodiment, details and constructions and their features described above and illustrated in the attached figures may be interchanged among the various embodiment while remaining within the scope of the invention. Additionally, it is understood that various modifications could be made to any of the elements described herein above while remaining within the scope of the invention.

1. A trabecular structure for a bone implant (102), wherein the trabecular structure comprises:
an augment (104, 304, 306), wherein the augment (104, 304, 306) is disposed on the bone implant (102), wherein the augment (104, 304, 306) comprises:
a wall (204) and;
a porous structure (202), wherein the porous structure (202) enclosed inside of the wall (204), wherein the porous structure (202) is made up of a plurality of cavities (214), wherein the plurality of cavities (214) are form by cross connecting a plurality of straight rib (216);
wherein the augment (104, 304, 306) enables a growth of the bone through the porous structure (202).

2. The trabecular structure for the bone implant (102) as claimed in claim 1, wherein the augment (104, 304, 306) comprises at least one threaded hole (108) for a fastener (106), wherein the fastener (106) is configured to constrain the motion of the augment (104, 304, 306) with respect to the bone implant (102).

3. The trabecular structure for the bone implant (102) as claimed in claim 1, wherein the augment (104, 304, 306) comprises a first surface (208) and a second surface (210), wherein the first surface (208) comes in contact with the bone implant (102) and the second surface (210) comes in contact with a cut surface of the bone.

4. The trabecular structure for the bone implant (102) as claimed in claim 1, wherein the augment (104, 304, 306) comprises an opening (110) for disposing the bone implant (102).

5. The trabecular structure for the bone implant (102) as claimed in claim 1, wherein the height of the augment (104, 304, 306) from the first surface (208) to the second surface (210) varies from 2 mm to 25 mm.

6. The trabecular structure for the bone implant (102) as claimed in claim 1, wherein the porosity of the porous structure (202) varies from 60 to 70%, wherein the thickness of the rib (216) is 0.2 mm.
